(19) United States
(12) Patent Application Publication
(10) Pub. No.: US 2018/0071296 A1
(43) Pub. Date: Mar. 15, 2018

(54) COMBINATION THERAPY COMPRISING AN INHIBITOR OF JAK, CDK, AND PIM

(71) Applicant: Novartis AG, Basel (CH)

(72) Inventors: Zhu Alexander Cao, Acton, MA (US); Maria PINZON-ORTIZ, Upton, MA (US); Xianhui RONG, Boxborough, MA (US)

(21) Appl. No.: 15/710,069

(22) Filed: Sep. 20, 2017

Related U.S. Application Data
(63) Continuation of application No. 15/039,869, filed on May 27, 2016, now abandoned.
(60) Provisional application No. 62/082,174, filed on Nov. 20, 2014.
(60) Provisional application No. 62/081,210, filed on Nov. 18, 2014.
(60) Provisional application No. 61/909,547, filed on Nov. 27, 2013.

(51) Int. Cl. A61K 31/519; A61K 31/444; A61K 45/06

(52) U.S. Cl. CPC A61K 31/519; A61K 31/444; A61K 2300/00; A61K 45/06

(57) ABSTRACT

The present invention relates to a pharmaceutical combination which comprises (a) a JAK inhibitor compound, (b) a CDK inhibitor, and (c) a PIM kinase inhibitor compound, and optionally, at least one pharmaceutically acceptable carrier for simultaneous, separate or sequential use, in particular for the treatment of a myeloid neoplasm or leukemia; a pharmaceutical composition comprising such a combination; the use of such a combination for the preparation of a medicament for the treatment of myeloid neoplasm or leukemia; a commercial package or product comprising such a combination as a combined preparation for simultaneous, separate or sequential use; and to a method of treatment of a mammal, especially a human.

### FIELD OF THE INVENTION

The present invention relates to a pharmaceutical combination comprising a JAK inhibitor, a CDK inhibitor and a PIM inhibitor for the treatment of cancer; the uses of such combinations in the treatment of cancer; and to a method of treating warm-blooded animals including humans suffering cancer comprising administering to said animal in need of such treatment an effective dose of a JAK inhibitor, a CDK inhibitor and a PIM inhibitor.

### BACKGROUND OF THE INVENTION

Cancer is the second leading cause of death in the United States. Although “cancer” is used to describe many different types of cancer, e.g., breast, prostate, lung, colon, and pancreatic, each type of cancer differs both at the phenotypic level and the genetic level. The unregulated growth characteristic of cancer occurs when the expression of one or more genes becomes disregulated due to mutations, and cell growth can no longer be controlled.

Myeloproliferative neoplasms (MPNs) are diseases that cause an overproduction of blood cells (platelets, white blood cells and red blood cells) in the bone marrow. MPNs include polycythernia vera (PV), primary or essential thrombocythemia (ET), primary or idiopathic myelofibrosis, chronic myelogenous (myelocytic) leukemia (CML), chronic neutrophilic leukemia (CNL), juvenile myelomonocytic leukemia (JML) and chronic eosinophilic leukemia (CEL)/hyper eosinophilic syndrome (HES). These disorders are grouped together because they share some or all of the following features: involvement of a multipotent hematopoietic progenitor cell, dominance of the transformed clone over the non-transformed hematopoietic progenitor cells, overproduction of one or more hematopoietic lineages in the absence of a definable stimulus, growth factor-independent colony formation in vitro, marrow hypercellularity, megakaryocyte hyperplasia and dysplasia, abnormalities predominantly involving chromosomes 1, 8, 9, 13, and 20, thrombotic and hemorrhagic diatheses, exuberant extramedullary hematopoiesis, and spontaneous transformation to acute leukemia or development of marrow fibrosis but at a low rate, as compared to the rate in CML. The incidence of MPNs varies widely, ranging from approximately 3 per 100,000 individuals older than 60 years annually for CML to 0.13 per 100,000 children from birth to 14 years annually for JML (Vardiman A N et al., Blood 100 (7): 2292-302, 2002). Accordingly, there remains a need for new treatments of MPNs, as well as other cancers such as solid tumors.

### SUMMARY OF THE INVENTION

The present invention relates to a pharmaceutical combination comprising (1) a first agent which is a JAK inhibitor or a pharmaceutically acceptable salt thereof, (2) a second agent which is a CDK inhibitor or a pharmaceutically acceptable salt thereof, and (3) a third agent that is a PIM inhibitor or a pharmaceutically acceptable salt thereof. More specifically, it relates to the treatment of solid tumors and hematological malignancies using the combination.

Such combination may be for simultaneous, separate or sequential use for the treatment of a cancer.

In one embodiment, the JAK inhibitor is ruxolitinib, which is also identified herein as Compound A, and has the chemical name of (3R)-3-cyclopentyl-3-[4-(7H-pyrrolo[2,3-d]pyrimidin-4-yl)pyrazol-1-yl]propanenitrile. Ruxolitinib is marketed under tradenames Jakafi® and Jakavia®.

In one embodiment, the CDK inhibitor is CDK4/6 inhibitor.

The CDK4/6 inhibitor can be, for example,

Compound B, described by Formula B below:

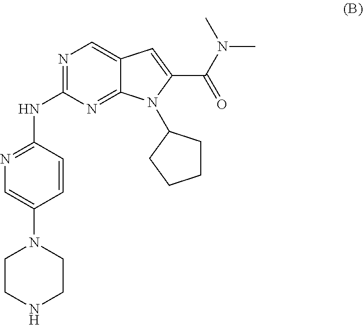 [SMILES: CN(C)C(=O)c1cc2cnc(Nc3ccc(N4CCNCC4)cn3)nc2n1C1CCCC1]

or pharmaceutically acceptable salt(s) thereof.

In one embodiment, the PIM inhibitor is Compound C (Chemical name: N-(4-((1R,3S,5S)-3-amino-5-methylcyclohexyl)pyridin-3-yl)-6-(2,6-difluorophenyl)-5-fluoropicolinamide), described by Formula C below:

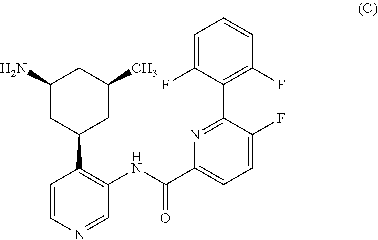 [SMILES: C[C@@H]1C[C@H](N)C[C@H](c2ccncc2NC(=O)c2ccc(F)c(-c3c(F)cccc3F)n2)C1]

or pharmaceutically acceptable salt(s) thereof.

The present invention further relates to the above pharmaceutical combination(s) for use in the treatment of a cancer.

The present invention further relates to a method for the treatment of a cancer comprising administering the above pharmaceutical combination(s) in jointly therapeutically effective amount, to a warm-blooded animal, preferably a human, in need thereof.

In accordance with the present invention, the compounds in the pharmaceutical combination(s) may be administered either as a single pharmaceutical composition, as separate compositions, or sequentially.

The present invention further relates to a kit comprising the pharmaceutical combination.

### BRIEF DESCRIPTION OF THE DRAWINGS

FIG. 1 shows the reduction of total tumor load at study endpoint in a murine MPN model, BA/F3-EpoR-JAK2^{V617F} with Compound A, Compound B and the combination. Data were collected with IVIS Spectrum Preclinical in vivo imaging system (Perkin Elmer).

FIG. 2 shows the reduction of spleen weight at study endpoint in the murine MPN model BA/F3-EpoR-JAK2^{V617F} with Compound A, Compound B monotherapies, and the combination of Compound A and Compound B.

FIG. 3 shows the modulation of JAK2V617F allele burden in PBMC at study endpoint in the murine MPN model BA/F3-EpoR-JAK2^{V617F} with Compound A, and the combination of Compound A and Compound B.

FIG. 4 shows the reduction of total tumor load at study endpoint in the murine MPN model BA/F3-EpoR-JAK2^{V617F} with Compound A, and the triple combination of Compound A, Compound B and Compound C. Data were collected with IVIS Spectrum Preclinical in vivo imaging system (Perkin Elmer).

FIG. 5 shows the reduction of spleen weight at study endpoint in the murine MPN model BA/F3-EpoR-JAK2^{V617F} with Compound A, and the combination of Compound A, Compound B and Compound C.

FIG. 6 shows the reduction of JAK2V617F allele burden in PBMC at study endpoint in the murine MPN model BA/F3-EpoR-JAK2^{V617F} with Compound A, and the combination of Compound A, Compound B and Compound C.

FIG. 7 shows the dose-sparing effect of each of Compound A, B or C on efficacy.

FIG. 8 shows the dose-sparing effect of all 3 agents (Compounds A, B and C) on efficacy.

FIG. 9 show the effects of “intermittent dosing” on efficacy.

### DETAILED DESCRIPTION OF THE INVENTION

The following general definitions are provided to better understand the invention.

### JAK Inhibitors

The JAK family plays a role in the cytokine-dependent regulation of proliferation and function of cells involved in immune response. Four mammalian JAK family members are: JAK1 (also known as Janus kinase-1), JAK2 (also known as Janus kinase-2), JAK3 (also known as Janus kinase, leukocyte; JAKL; L-JAK and Janus kinase-3) and TYK2 (also known as protein-tyrosine kinase 2). Aberrant JAK-STAT signaling has been implicated in multiple human pathogenesis. The genetic aberration of JAK2 and the associated activation of STAT in myeloproliferative neoplasia (MPN) is one example of the involvement of this pathway in human neoplasia. Mutation in the upstream thrombopoietin receptor (MPLW525L) and the loss of JAK regulation by LNK (exon 2) have been associated with myelofibrosis (Vainchenker W et al., Blood 2011; 118:1723; Pikman Y et al., Plox Med. 2006, 3: e270). Mutation in JAK2, mostly JAK2 V617F, that leads to constitutive activation of JAK2, have been noted in the majority of patients with primary myelofibrosis (Kralovics R et al., N Engl. J Med 2005, 352; 1779; Baxter E J et al., Lancet 2005, 365: 1054; Levine R L et al., Cancer Cell 2005, 7: 387). Additional mutations in JAK2 exon 12 have been identified in polycythernia vera and idiopathic erythrocytosis (Scott L M et al., N Engl J Med 2007, 356: 459). Additionally, activated JAK-STAT has been suggested as a survival mechanism for human cancers (Hedvat M et al., Cancer Cell 2009; 16: 487). Recently, data have emerged to indicate that JAK2/STAT5 inhibition would circumvent resistant to PI3K/mTOR blockade in metastatic breast cancer (Britschgi A et al., Cancer Cell 2012; 22: 796). Also, the use of a JAK1/2 inhibitor in IL-6-driven breast, ovarian, and prostate cancers has led to the inhibition of tumor growth in preclinical models (Sansone P and Bromberg J; J. Clinical Oncology 2012, 30: 1005).

### CDK Inhibitors

Tumor development is closely associated with genetic alteration and deregulation of CDKs and their regulators, suggesting that inhibitors of CDKs may be useful anti-cancer therapeutics. Indeed, early results suggest that transformed and normal cells differ in their requirement for, e.g., cyclin D/CDK4/6 and that it may be possible to develop novel antineoplastic agents devoid of the general host toxicity observed with conventional cytotoxic and cytostatic drugs.

The function of CDKs is to phosphorylate and thus activate or deactivate certain proteins, including e.g. retinoblastoma proteins, lamins, histone H1, and components of the mitotic spindle. The catalytic step mediated by CDKs involves a phospho-transfer reaction from ATP to the macromolecular enzyme substrate. Several groups of compounds (reviewed in e.g. Fischer, P. M. Curr. Opin. Drug Discovery Dev. 2001, 4, 623-634) have been found to possess anti-proliferative properties by virtue of CDK-specific ATP antagonism.

At a molecular level mediation of CDK/cyclin complex activity requires a series of stimulatory and inhibitory phosphorylation, or dephosphorylation, events. CDK phosphorylation is performed by a group of CDK activating kinases (CAKs) and/or kinases such as wee1, Myt1 and Mik1. Dephosphorylation is performed by phosphatases such as cdc25(a & c), pp2a, or KAP.

CDK/cyclin complex activity may be further regulated by two families of endogenous cellular proteinaceous inhibitors: the Kip/Cip family, or the INK family. The INK proteins specifically bind CDK4 and CDK6. p16ink4 (also known as MTS1) is a potential tumour suppressor gene that is mutated, or deleted, in a large number of primary cancers. The Kip/Cip family contains proteins such as p21Cip1, Waf1, p27Kip1 and p57kip2, where p21 is induced by p53 and is able to inactivate the CDK2/cyclin(E/A) complex. Atypically low levels of p27 expression have been observed in breast, colon and prostate cancers. Conversely over expression of cyclin E in solid tumours has been shown to correlate with poor patient prognosis. Over expression of cyclin D1 has been associated with oesophageal, breast, squamous, and non-small cell lung carcinomas.

The pivotal roles of CDKs, and their associated proteins, in co-ordinating and driving the cell cycle in proliferating cells have been outlined above. Some of the biochemical pathways in which CDKs play a key role have also been described. The development of monotherapies for the treatment of proliferative disorders, such as cancers, using therapeutics targeted generically at CDKs, or at specific CDKs, is therefore potentially highly desirable. Thus, there is a continued need to find new therapeutic agents to treat human diseases.

### PIM Inhibitors

The PIM proteins (Proviral Integration site for the Moloney-murine leukemia virus) are a family of three ser/thr kinases, with no regulatory domains in their sequences and are considered as constitutively active upon their translation (Qian, K. C., et al. J. Biol. Chem. 2004. p6130-6137). They are oncogenes involved in the regulation of cell cycle, proliferation, apoptosis and drug resistance (Mumenthaler et al, Mol Cancer Ther. 2009; p2882). Their expression is found particularly elevated in hematopoietic cancers, but some reports have shown an over-expression of PIM1 in pancreatic, prostate and liver cancers as well as a PIM3 expression in certain solid tumors (Reviewed by Alvarado et al, Expert Rev. Hematol. 2012, p81-96). PIM kinases are regulated by rates of transcription, translation and proteasomal degradation, but the factors that dictate these events are still poorly understood. One pathway that is well established and known to induce PIM1/2 expression is the JAK/STAT signaling pathway (Miura et al, Blood. 1994, p4135-4141). The STAT proteins are transcription factors, activated downstream of the JAK tyrosine kinases, upon cell surface receptor interaction with their ligands, such as cytokines. Both STAT3 and STAT5 are known to bind to the PIM promoter to induce PIM expression (Stout et al. J Immunol, 2004; 173:6409-6417). Beside the JAK/STATs, the VEGF pathway was also shown to up-regulate PIM expression in endothelial cells during angiogenesis of the ovary, and in human umbilical cord vein cells (Zipo et al, Nat Cell Biol. 2007, p932-944).

It has been discovered that administering a JAK inhibitor, a CDK inhibitor, and a PIM inhibitor combination of the invention provides synergistic effects for treating proliferative diseases of the blood, which can include can myeloid neoplasm, leukemia, other cancers of the blood and could be potentially useful in treating solid cancers as well. Such an approach—combination or co-administration of the two types of agents—can be useful for treating individuals suffering from cancer who do not respond to or are resistant to currently-available therapies. The combination therapy provided herein is also useful for improving the efficacy and/or reducing the side effects of currently-available cancer therapies for individuals who do respond to such therapies.

“Combination” refers to either a fixed combination in one dosage unit form, or a non-fixed combination (or kit of parts) for the combined administration where a compound and a combination partner (e.g. another drug as explained below, also referred to as “therapeutic agent” or “co-agent”) may be administered independently at the same time or separately within time intervals, especially where these time intervals allow that the combination partners show a cooperative, e.g. synergistic effect. The term “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “fixed combination” means that the active ingredients, e.g. a compound of formula A and a combination partner, are both administered to a patient simultaneously in the form of a single entity or dosage. The terms “non-fixed combination” or “kit of parts” mean that the active ingredients, e.g. a compound of formula A and a combination partner, are both administered to a patient as separate entities either simultaneously, concurrently or sequentially with no specific time limits, wherein such administration provides therapeutically effective levels of the two compounds in the body of the patient.

“Treatment” includes prophylactic and therapeutic treatment (including but not limited to palliative, curing, symptom-alleviating, symptom-reducing) as well as the delay of progression of a cancer disease or disorder. The term “prophylactic” means the prevention of the onset or recurrence of a cancer. The term “delay of progression” as used herein means administration of the combination to patients being in a pre-stage or in an early phase of the cancer to be treated, a pre-form of the corresponding cancer is diagnosed and/or in a patient diagnosed with a condition under which it is likely that a corresponding cancer will develop.

“Pharmaceutical preparation” or “pharmaceutical composition” refers to a mixture or solution containing at least one therapeutic agent to be administered to a warm-bloodeded, e.g., a human.

“Co-administer”, “co-administration” or “combined administration” or the like are meant to encompass administration of the selected therapeutic agents to a single patient, and are intended to include treatment regimens in which the agents are not necessarily administered by the same route of administration or at the same time.

“Pharmaceutically acceptable” refers to those compounds, materials, compositions and/or dosage forms, which are, within the scope of sound medical judgment, suitable for contact with the tissues of mammals, especially humans, without excessive toxicity, irritation, allergic response and other problem complications commensurate with a reasonable benefit/risk ratio.

“Therapeutically effective” preferably relates to an amount of a therapeutic agent that is therapeutically or in a broader sense also prophylactically effective against the progression of a cancer.

“Jointly therapeutically effective” means that the therapeutic agents may be given separately (in a chronologically staggered manner, especially a sequence-specific manner) in such time intervals that they prefer, in the warm-blooded animal, especially human, to be treated, still show a (preferably synergistic) interaction. Whether this is the case can, inter alia, be determined by following the blood levels, showing that both compounds are present in the blood of the human to be treated at least during certain time intervals.

“Single pharmaceutical composition” refers to a single carrier or vehicle formulated to deliver effective amounts of both therapeutic agents to a patient. The single vehicle is designed to deliver an effective amount of each of the agents, along with any pharmaceutically acceptable carriers or excipients. In some embodiments, the vehicle is a tablet, capsule, pill, or a patch. In other embodiments, the vehicle is a solution or a suspension.

“Dose range” refers to an upper and a lower limit of an acceptable variation of the amount of therapeutic agent specified. Typically, a dose of the agent in any amount within the specified range can be administered to patients undergoing treatment.

“Subject”, “patient”, or “warm-blooded animal” is intended to include animals. Examples of subjects include mammals, e.g., humans, dogs, cows, horses, pigs, sheep, goats, cats, mice, rabbits, rats, and transgenic, non-human animals. In certain embodiments, the subject is a human, e.g., a human suffering from, at risk of suffering from, or potentially capable of suffering from a brain tumor disease. Particularly preferred, the subject or warm-blooded animal is human.

The terms “about” or “approximately” usually means within 20%, more preferably within 10%, and most preferably still within 5% of a given value or range. Alternatively, especially in biological systems, the term “about” means within about a log (i.e., an order of magnitude) preferably within a factor of two of a given value.

The present invention relates to a pharmaceutical combination comprising (1) a CDK inhibitor or a pharmaceutically acceptable salt thereof and (2) a mTOR inhibitor or a pharmaceutically acceptable salt thereof.

Such combination may be for simultaneous, separate or sequential use for the treatment of a cancer.

In one embodiment, the JAK inhibitor is ruxolitinib, which is also identified herein as Compound A, and has the chemical name of (3R)-3-cyclopentyl-3-[4-(7H-pyrrolo[2,3-d]pyrimidin-4-yl)pyrazol-1-yl]propanenitrile. Ruxolitinib is marketed under tradenames Jakafi® and Jakavi®.

In one embodiment, the CDK inhibitor is CDK4/6 inhibitor.

The CDK4/6 inhibitor can be, for example,

Compound B (Chemical name: 7-Cyclopentyl-2-(5-piperazin-1-yl-pyridin-2-ylamino)-7H-pyrrolo[2,3-d]pyrimidine-6-carboxylic acid dimethylamide), described by Formula B below:

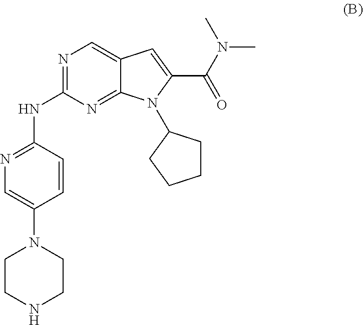 [SMILES: CN(C)C(=O)c1cc2cnc(Nc3ccc(N4CCNCC4)cn3)nc2n1C1CCCC1]

or pharmaceutically acceptable salt(s) thereof.

In one embodiment, the PIM inhibitor is Compound C (Chemical name: N-(4-((1R,3S,5S)-3-amino-5-methylcyclohexyl)pyridin-3-yl)-6-(2,6-difluorophenyl)-5-fluoropicolinamide), described by Formula C below:

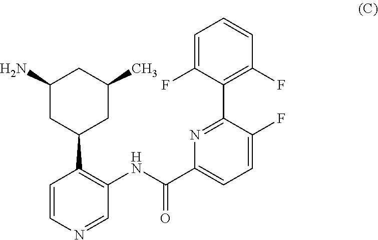 [SMILES: C[C@@H]1C[C@H](N)C[C@H](c2ccncc2NC(=O)c2ccc(F)c(-c3c(F)cccc3F)n2)C1]

or pharmaceutically acceptable salt(s) thereof.

The present invention further relates to the above pharmaceutical combination(s) for use in the treatment of a cancer.

The present invention further relates to a method for the treatment of a cancer comprising administering the above pharmaceutical combination(s) in jointly therapeutically effective amount, to a warm-blooded animal, preferably a human, in need thereof.

In accordance with the present invention, the compounds in the pharmaceutical combination(s) may be administered either as a single pharmaceutical composition, as separate compositions, or sequentially.

The present invention further relates to a kit comprising the pharmaceutical combination.

The Compounds A, B and C can be synthesized by one skilled in the art. Specifically, Compound A is disclosed in U.S. Pat. No. 7,598,257; Compound B is disclosed as Example 74 of WO2010/020675; and Compound C is disclosed in WO 2010/026124 as Example 70.

Comprised are likewise the pharmaceutically acceptable salts thereof, the corresponding racemates, diastereoisomers, enantiomers, tautomers, as well as the corresponding crystal modifications of above disclosed compounds where present, e.g. solvates, hydrates and polymorphs, which are disclosed therein. The compounds used as active ingredients in the combinations of the present invention can be prepared and administered as described in the cited documents, respectively. Also within the scope of this invention is the combination of more than two separate active ingredients as set forth above, i.e., a pharmaceutical combination within the scope of this invention could include three active ingredients or more.

It is believed that the combination(s) of the present invention possesses beneficial therapeutic properties, e.g. synergistic interaction, strong in vitro or in vivo anti-proliferative activity and/or strong in vitro or in vivo antitumor response, which render it particularly useful for the treatment of cancer.

Provided herein are methods of treating cancer, e.g., myeloproliferative neoplasms and solid tumors, using the combination therapy treatment described above.

As used herein, “cancer” refers to any disease that is caused by or results in inappropriately high levels of cell division, inappropriately low levels of apoptosis, or both. Examples of cancer include, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors.

Furthermore, the combination therapy provided herein relates to a pharmaceutical composition for treatment of solid or liquid tumors in warm-blooded animals, including humans, comprising and antitumor-effective dose of a compounds of the combination as described above.

The combination therapy provided herein can be used in the treatment of solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangio sarcoma, lvmphangioendotheliosarcoma, synovioma, mesothelioma, Ewing's tumor, leiomyo sarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, crailiopharyngioma, ependymoma, pinealoma, hemangioblastoma, acoustic neuroma, oligodenroglioma, schwamioma, meningioma, melanoma, neuroblastoma, and retinoblastoma).

In a certain embodiment, the cancer that can be treated using the combination provided herein is a myeloproliferative disoder. Myeloproliferative disorders (MPDS), now commonly referred to as meyloproliferative neoplasms (MPNs), are in the class of haematological malignancies that are clonal disorders of hematopoietic progenitors. Tefferi, A. and Vardiman, J. W., Classification and diagnosis of myeloproliferative neoplasms: The 2008 World Health Organization criteria and point-of-care diagnostic algorithms, Leukemia, September 2007, 22: 14-22, is hereby incorporated by reference. They are characterized by enhanced proliferation and survival of one or more mature myeloid lineage cell types. This category includes but is not limited to, chronic myeloid leukemia (CML), polycythemia vera (PV), essential thrombocythemia (Er), primary or idiopathic myelofibrosis (PMF), chronic neutrophilic leukemia, chronic eosinophilic leukemia, chronic myelomonocytic leukemia, juvenile myelomonocytic leukemia, hypereosinophilic syndrome, systemic mastocytosis, and atypical chronic myelogenous leukemia. Tefferi, A. and Gilliland, D. G., Oncogenes in myeloproliferative disorders, Cell Cycle. March 2007, 6(5): 550-566 is hereby fully incorporated by reference in its entirety for all purposes.

It is one objective of this invention to provide a pharmaceutical composition comprising a quantity, which is jointly therapeutically effective at targeting or preventing a cancer, of each therapeutic agent of the invention.

In accordance with the present invention, agents in the composition of the present invention may be administered together in a single pharmaceutical composition, separately in two or more separate unit dosage forms, or sequentially. The unit dosage form may also be a fixed combination.

The pharmaceutical compositions for separate administration of agents or for the administration in a fixed combination (i.e., a single galenical composition comprising at least two therapeutic agents according to the invention may be prepared in a manner known per se and are those suitable for enteral, such as oral or rectal, topical, and parenteral administration to subjects, including mammals (warm-blooded animals) such as humans, comprising a therapeutically effective amount of at least one pharmacologically active combination partner alone, e.g., as indicated above, or in combination with one or more pharmaceutically acceptable carriers or diluents, especially suitable for enteral or parenteral application. Suitable pharmaceutical compositions contain, e.g., from about 0.1% to about 99.9%, preferably from about 1% to about 60%, of the active ingredient(s).

Pharmaceutical compositions for the combination therapy for enteral or parenteral administration are, e.g., those in unit dosage forms, such as sugar-coated tablets, tablets, capsules or suppositories, ampoules, injectable solutions or injectable suspensions. Topical administration is e.g. to the skin or the eye, e.g. in the form of lotions, gels, ointments or creams, or in a nasal or a suppository form. If not indicated otherwise, these are prepared in a manner known per se, e.g., by means of conventional mixing, granulating, sugar-coating, dissolving or lyophilizing processes. It will be appreciated that the unit content of each agent contained in an individual dose of each dosage form need not in itself constitute an effective amount since the necessary effective amount can be reached by administration of a plurality of dosage units.

Pharmaceutical compositions may comprise one or more pharmaceutical acceptable carriers or diluents and may be manufactured in conventional manner by mixing one or both combination partners with a pharmaceutically acceptable carrier or diluent. Examples of pharmaceutically acceptable diluents include, but are not limited to, lactose, dextrose, mannitol, and/or glycerol, and/or lubricants and/or polyethylene glycol. Examples of pharmaceutically acceptable acceptable binders include, but are not limited to, magnesium aluminum silicate, starches, such as corn, wheat or rice starch, gelatin, methylcellulose, sodium carboxymethylcellulose and/or polyvinylpyrrolidone, and, if desired, pharmaceutically acceptable disintegrators include, but are not limited to, starches, agar, alginic acid or a salt thereof, such as sodium alginate, and/or effervescent mixtures, or adsorbents, dyes, flavorings and sweeteners. It is also possible to use the compounds of the present invention in the form of parenterally administrable compositions or in the form of infusion solutions. The pharmaceutical compositions may be sterilized and/or may comprise excipients, for example preservatives, stabilizers, wetting compounds and/or emulsifiers, solubilisers, salts for regulating the osmotic pressure and/or buffers.

In particular, a therapeutically effective amount of each of the combination partner of the combination of the invention may be administered simultaneously or sequentially and in any order, and the components may be administered separately or as a fixed combination. For example, the method of preventing or treating a cancer according to the invention may comprise: (i) administration of the first agent in free or pharmaceutically acceptable salt form; and (ii) administration of a second agent in free or pharmaceutically acceptable salt form, simultaneously or sequentially in any order, in jointly therapeutically effective amounts, preferably in synergistically effective amounts, e.g., in daily or intermittently dosages corresponding to the amounts described herein. The individual combination partners of the combination of the invention may be administered separately at different times during the course of therapy or concurrently in divided or single combination forms. Furthermore, the term administering also encompasses the use of a pro-drug of a combination partner that convert in vivo to the combination partner as such. The instant invention is therefore to be understood as embracing all such regimens of simultaneous or alternating treatment and the term “administering” is to be interpreted accordingly.

The effective dosage of each of combination partner agents employed in the combination of the invention may vary depending on the particular compound or pharmaceutical composition employed, the mode of administration, the condition being treated, the severity of the condition being treated. Thus, the dosage regimen of the combination of the invention is selected in accordance with a variety of factors including type, species, age, weight, sex and medical condition of the patient; the severity of the condition to be treated; the route of administration; the renal and hepatic function of the patient; and the particular compound employed. A physician, clinician or veterinarian of ordinary skill can readily determine and prescribe the effective amount of the drug required to prevent, counter or arrest the progress of the condition. Optimal precision in achieving concentration of drug within the range that yields efficacy requires a regimen based on the kinetics of the drug's availability to target sites. This involves a consideration of the distribution, equilibrium, and elimination of a drug.

In various embodiments in human, the dose of Compound A can be 5 mg BID, 7.5 mg BID, 10 mg BID, 12.5 mg BID, 15 mg BID, 20 mg BID or 25 mg BID.

In various embodiments in human, the dose of Compound A can be 5 mg QD, 7.5 mg OD, 10 mg OD, 12.5 mg QD, 15 mg QD, 20 mg QD or 25 mg QD.

In various embodiments in human, the dose of Compound B can be 50 mg QD, 75 mg QD, 100 mg QD, 125 mg QD, 150 mg OD, 175 mg QD, or 200 mg OD.

In various embodiments in human, the dose of Compound C can be 50 mg OD, 75 mg QD, 100 mg QD, 125 mg QD, 150 mg QD, 175 mg QD, 200 mg QD, 250 mg OD, 300 mg QD or 350 mg QD.

In another embodiment in human, the dose of Compound A is 10-20 mg BID, the dose of Compound B is 100-200 mg QD and the dose of Compound C is 150-300 mg QD.

In another embodiment in human, the dose of Compound A is 15 rug BID, the dose of Compound B is 100 mg OD and the dose of Compound C is 250 mg OD.

In one embodiment in human, the dose of Compound A is 25-30 mg QD, the dose of Compound B is 100 mg OD and the dose of Compound C is 250 mg QD.

In one embodiment in human, Compound A is administered at a dose of 10-20 mg BID, Compound C is administered at a dose of 150-300 mg and Compound B is administered intermittently at a dose of 100-200 mg OD, e.g., Compound B may be administered daily for a specified period of time and then discontinued for a specified period of time and then administered again for a specified period of time.

In one embodiment, Compound B is administered daily for 21 days followed by no administration for 7 days and then administered again for 21 days followed by no administration for 7 days, etc.

A further benefit is that lower doses of the active ingredients of the combination of the invention can be used, e.g., that the dosages need not only often be smaller but are also applied less frequently, or can be used in order to diminish the incidence of side effects. This is in accordance with the desires and requirements of the patients to be treated.

The combination of the agents can be combined in the same pharmaceutical preparation or in the form of combined preparations “kit of parts” in the sense that the combination partners can be dosed independently or by use of different fixed combinations with distinguished amounts of the combination partners, i.e., simultaneously or at different time points. The parts of the kit of parts can then, e.g., be administered simultaneously or chronologically staggered, that is at different time points and with equal or different time intervals for any part of the kit of parts.

The present invention further relates to a kit comprising a first compound that is Compound A or pharmaceutically acceptable salts thereof, a second compound that is Compound B or pharmaceutically acceptable salts thereof, and a package insert or other labeling including directions for treating a cancer.

The following Examples illustrate the invention described above; they are not, however, intended to limit the scope of the invention in any way. The beneficial effects of the pharmaceutical combination of the present invention can also be determined by other test models known as such to the person skilled in the pertinent art.

The combination of Compound A, Compound B and Compound C was examined in a mouse model of MPN. In this model, Ba/F3 cells harbored Epo Receptor and JAK2 V617F mutations. Ba/F3-EpoR-JAK2^{V617F} was engineered with a luciferase tag for experimental imaging. Female SCID/Beige mice were inoculated with 1x10e6 Ba/F3-EpoR-JAK2^{V617F} cells through the tail vein. Total tumor load was monitored with IVIS xenogen technology. It is defined as the sum of dorsal and ventral photon signal. Additionally, JAK2V617F allele burden, defined as the relative ratio of JAK2V617F over wild type JAK2, is measured by taqman in PBMCs at study endpoint.

### EXAMPLE

In the first experiment, disease-bearing mice were randomized into treatment cohorts, based on the disease burden. Mice were treated with vehicle, Compound B at 75 mg/kg, by oral gavage (PO) daily (QD), Compound A at 60 mg/kg, PO, twice daily (BID) and the combination of both agents. At study endpoint, spleen weight from each of the study cohorts was obtained. Relative change in the spleen weight was calculated by normalizing individual spleen weight against the mean spleen weight of the cohort receiving vehicle treatment. The combination of Compound A and Compound B resulted in greater reduction in the disease burden and the spleen weight.

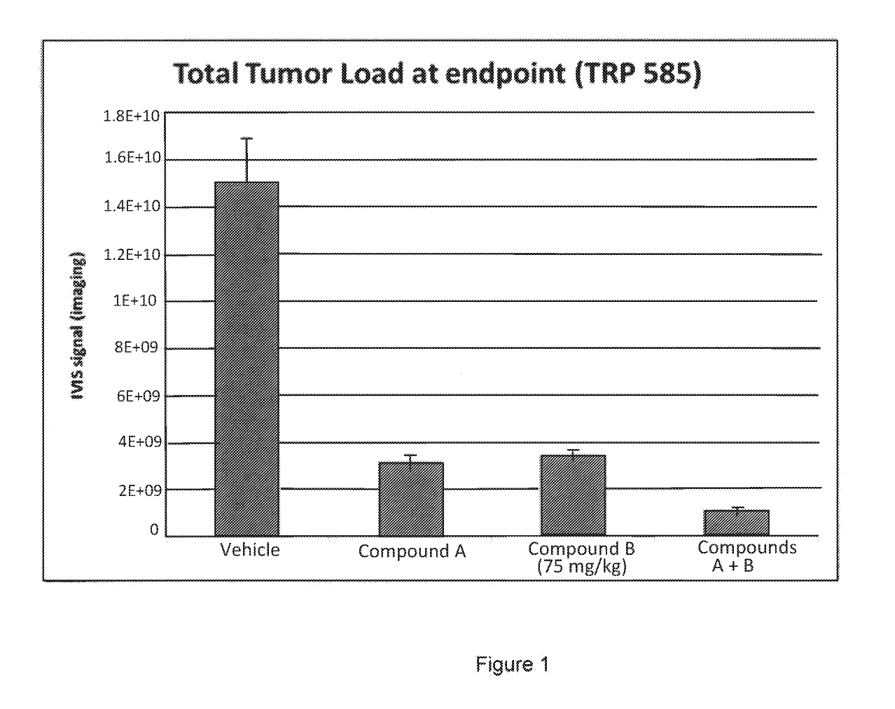

FIG. 1, the total tumor load, measured by the level of bioluminescene, was reduced with Compound A and Compound B monotherapy by ˜79% and ˜77%, respectively, relative to the vehicle control. It was reduced by ˜92% with the combination of Compound A and Compound B.

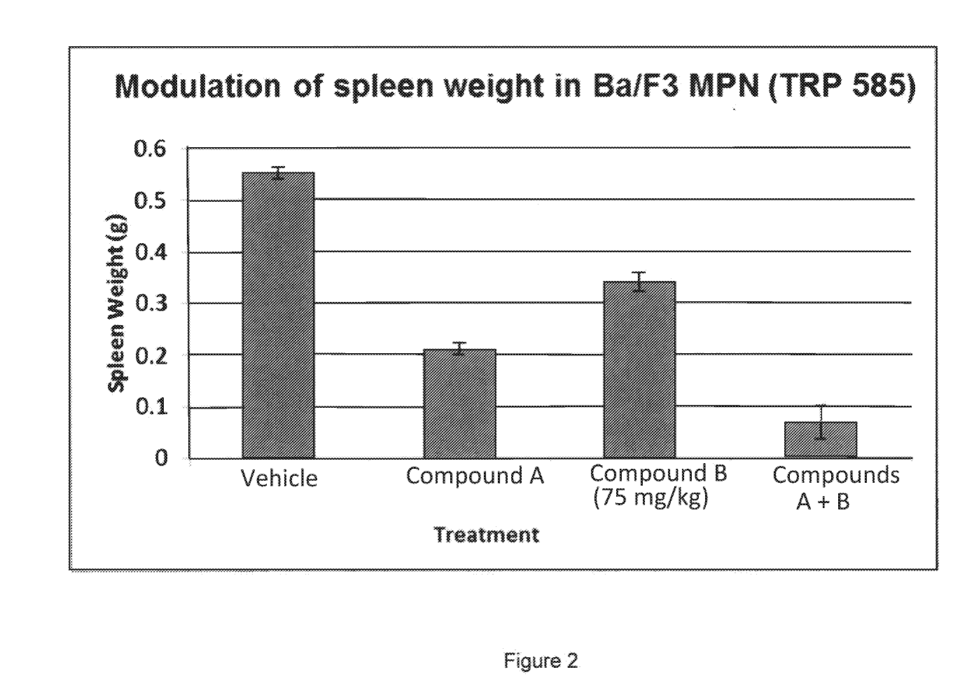

FIG. 2 shows the effects of Compound A and the combination of Compound A with Compound B on spleen weight in the MPN preclinical model. Compound A and Compound B monotherapies resulted in ˜62% and ˜38% reduction of spleen weight, respectively, relative to that of the vehicle control. The combination of Compound A and Compound B lead to ˜88% reduction of spleen weight, relative to that of the vehicle control.

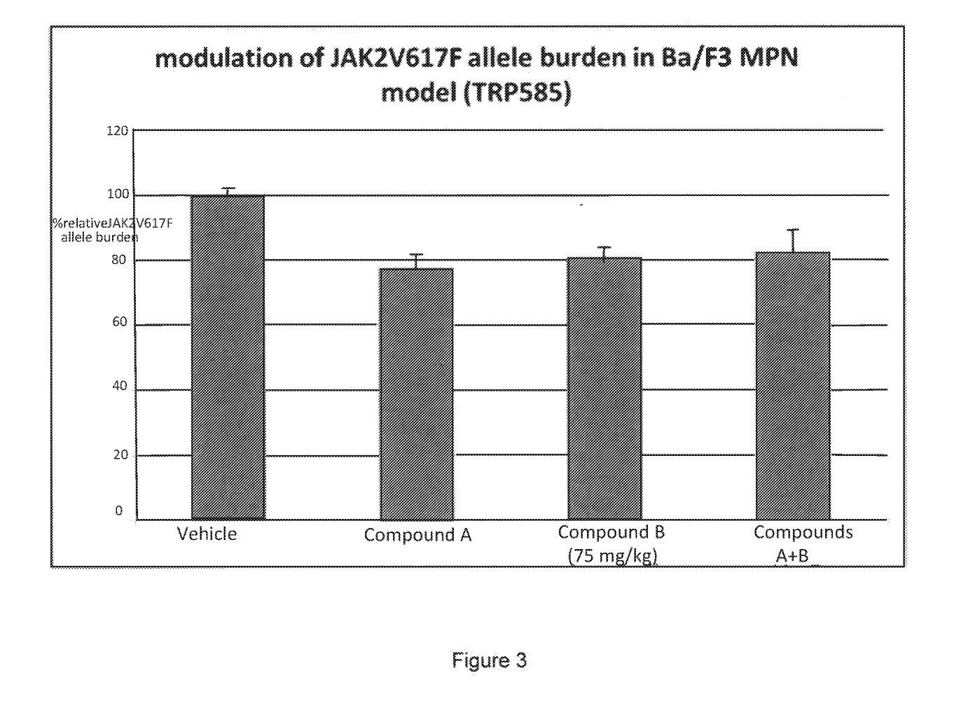

FIG. 3 shows the modulation of JAK2V617F allele burden in this model. Compound A, Compound B monotherapies and the combination all have comparable effects (˜18-22% reduction, relative to the vehicle control) on JAK2V617F allele burden.

### Example 2

In the second experiment, disease-bearing mice were randomized into treatment cohort, based on the disease burden. Mice were treated with vehicle, Compound A at 60 mg/kg, PO, twice daily (BID), and the triple combination of Compound A, Compound B (at 75 mg/kg, QD, PO) and Compound C (at 25 mg/kg, QD, PO). At study endpoint, spleen weight from each of the study cohorts was obtained. Relative spleen weight was calculated by normalizing individual spleen weight against the mean spleen weight of the cohort receiving vehicle treatment. The combination of Compound A, Compound B and Compound C resulted in more pronounced reduction in total tumor load and spleen weight. Also, the triple combination achieved notable reduction in the JAK2V617F allele burden in this model.

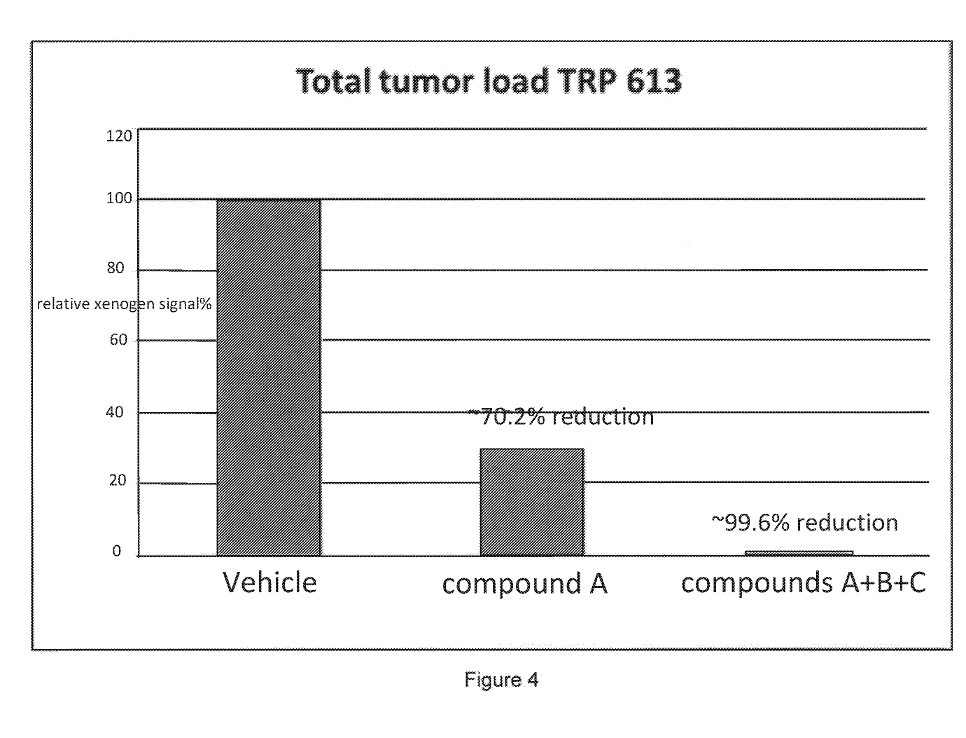

In FIG. 4, the total tumor load, measured by the level of bioluminescene, was reduced with Compound A treatment by ˜70%. The triple combination of Compound A, Compound B and Compound C reduced the total tumor burden by over 99%.

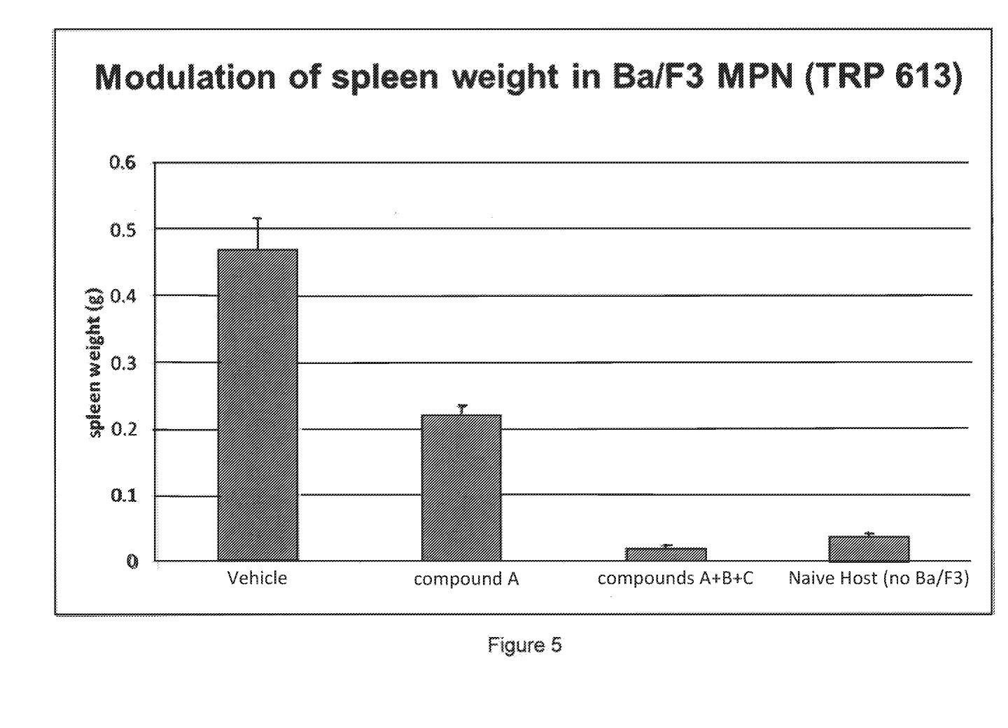

FIG. 5 shows the effects of Compound A and the triple combination of Compound A with Compound B and Compound C on spleen weight in the MPN preclinical model. Compound A monotherapy resulted in ˜53% reduction of spleen weight, relative to that of the vehicle control. The triple combination of Compound A, Compound B and Compound C lead to ˜96% reduction of spleen weight, relative to that of the vehicle control. The resulting spleen weight is similar to that in non-tumor-bearing, naive mice.

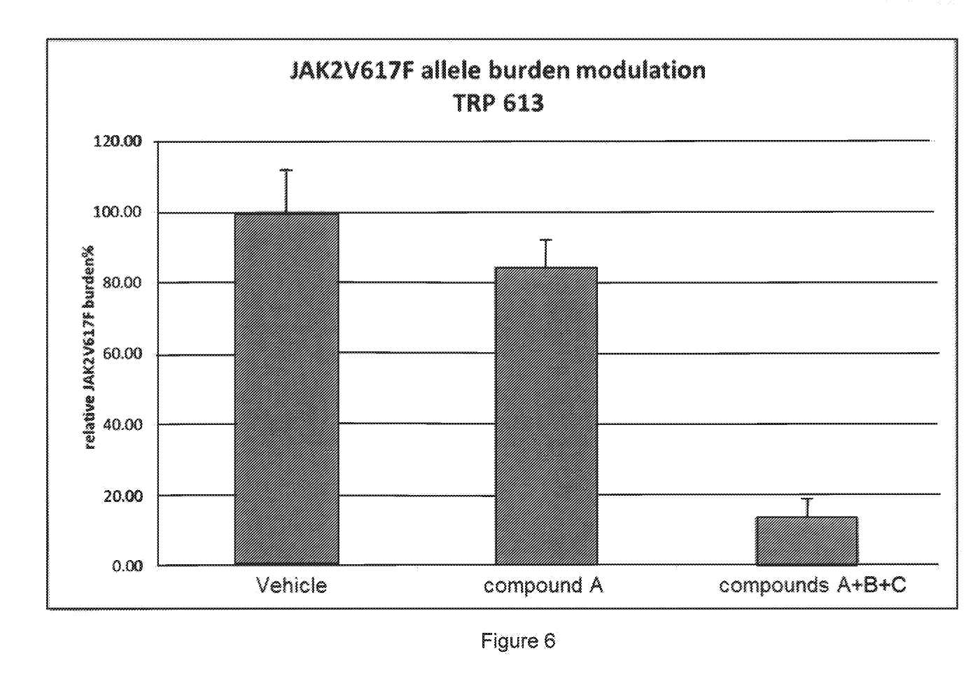

FIG. 6 shows the modulation of JAK2V617F allele burden in this model. Compound A monotherapy down-modulated allele burden by ˜15%. The triple combination of Compound A, Compound B, and Compound C down-modulated the JAK2V617F allele burden by ˜86%.

### Example 3

In this experiment, we aim to evaluate the efficacy when one agent of Compounds A, B and C is dose reduced. Disease-bearing mice were randomized into treatment cohorts, based on the disease burden. Mice were treated according to the following doses:

| | Compound | Compound | Compound |
|---|---|---|---|
| | A (BID) | C (QD) | B (QD) |
| Full dose triple (mouse) | 60 mg/kg | 25 mg/kg | 75 mg/kg |
| Triple @ 50% | 30 mg/kg | 25 mg/kg | 75 mg/kg |
| Compound A (mouse) | | | |
| Triple @ 50% | 60 mg/kg | 12.5 mg/kg | 75 mg/kg |
| Compound C (mouse) | | | |
| Triple @ 50% | 60 mg/kg | 25 mg/kg | 37.5 mg/kg |
| Compound B (mouse) | | | |
| Triple @ 50% | 30 mg/kg | 12.5 mg/kg | 37.5 mg/kg |
| Compounds A, B and C | | | |
| (mouse) | | | |

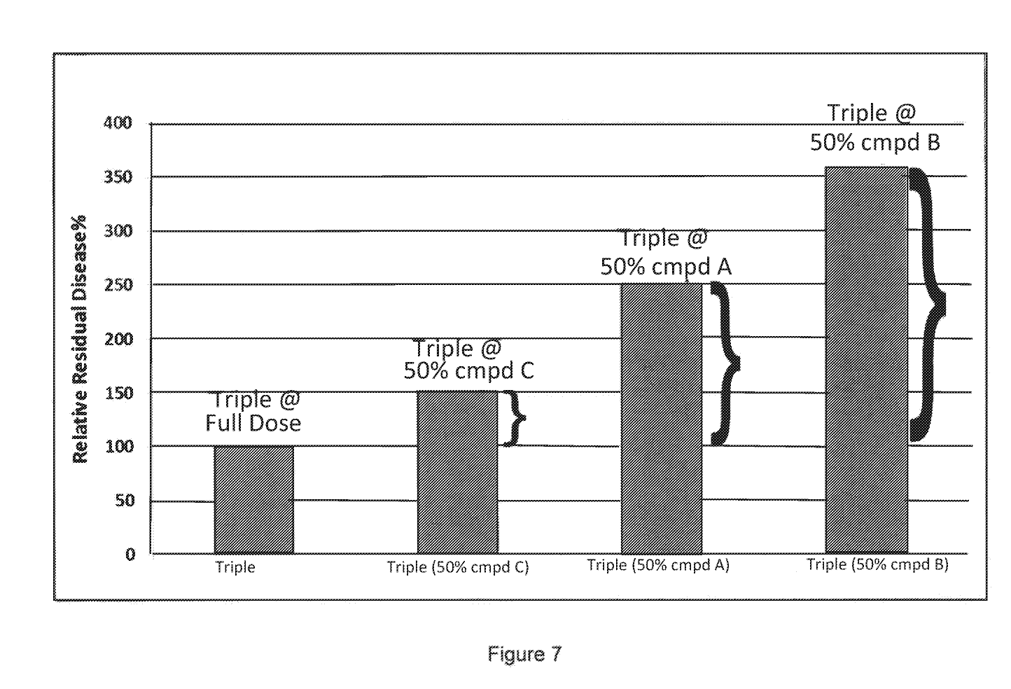

FIG. 7 shows that dose reduction of Compound C (from 25 mg/kg) has the least effect on efficacy and that dose reduction of Compound B (from 75 mg/kg) greatly impact efficacy.

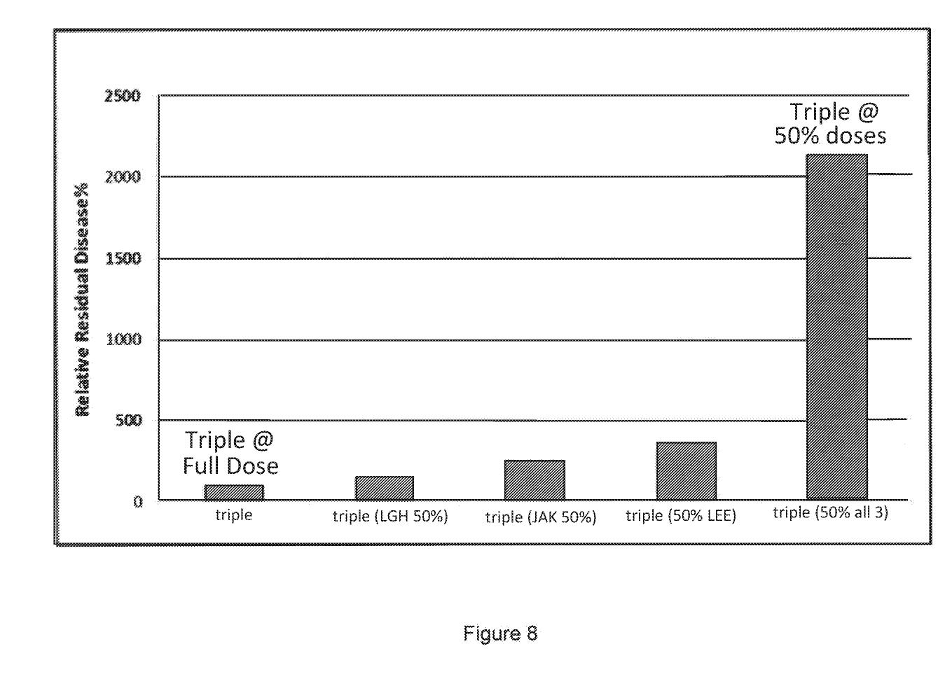

FIG. 8 shows that simultaneous dose reduction on all 3 agents has profound effect on efficacy.

Residual disease is the xenogen signal (remaining disease) when hosts are treated under the full-dose triple combination.

### Example 4

In this experiment, we aim to evaluate the efficacy on “intermittent dosing” schedule. Disease-bearing mice were randomized into treatment cohorts, based on the disease burden. Mice were treated according to the following doses:

| | Compound | Compound | Compound |
|---|---|---|---|
| | A (BID) | C (QD) | B (QD) |
| Full dose triple (mouse) | 60 mg/kg | 25 mg/kg | 75 mg/kg |
| Triple, Compound B at | 60 mg/kg | 25 mg/kg | 150 mg/kg |
| 2x/week (mouse) | | | (2x/week) |
| Triple, Compound C at | 60 mg/kg | 50 mg/kg | 75 mg/kg |
| 2x/week (mouse) | | (2x/week) | |

Residual disease is the xenogen signal (remaining disease) when hosts are treated under the full-dose triple combination.

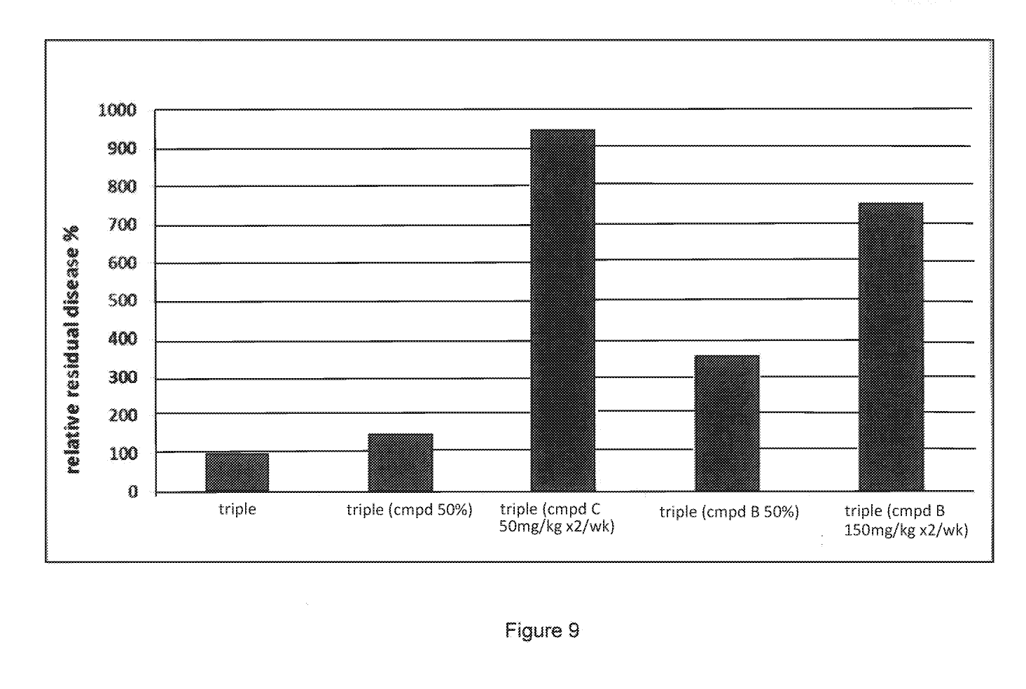

FIG. 9 shows that intermittent dosing has lead to clear reduction in efficacy in a BaF3 model. In the BaF/JAK2^{V617F} model, “intermittent dosing” in Compounds A-B-C triple combination leads to profound reduction of efficacy.

### Example 5

A phase Ib, multi-center, open label, dose-escalation study of combination of Compound A (ruxolitinib) and/or Compound B and/or Compound C administered orally in patients with myelofibrosis is planned.

Compound A is to be administered orally. The dose of Compound A can be 5 mg BID, 7.5 mg BID, 10 mg BID, 12.5 mg BID, or 15 mg BID.

Compound B is to be administered orally. The dose of Compound B can be 50 mg QD, 75 mg QD, 100 mg QD, 125 mg QD, 150 mg QD, 175 mg QD or 200 mg QD.

Compound C is to be administered orally. The dose of Compound C can be 50 mg QD, 75 mg QD, 100 mg QD, 125 mg QD, 150 mg QD, 175 mg QD or 200 mg QD.

The main objective of the trial is to estimate the MTD and/or RDE for each of the following three treatment arms in patients with myelofibrosis: (1) Compound C+Compound A; (2) Compound B+Compound A; and (3) Compound A+Compound B+Compound C.

The secondary objectives are: (1) to characterize the safety and tolerability of Compound C+Compound A, Compound B+Compound A, and the triple combination of Compound A+Compound B+Compound C; (2) to assess preliminary anti-myelofibrosis activity of Compound C+Compound A, Compound B+Compound A, and the triple combination of Compound A+Compound B+Compound C; and (3) to characterize the PK profiles of combination of Compound A, Compound B and Compound C

We claim:

1. A pharmaceutical combination comprising
(a) Ruxolitinib (Compound A) or a pharmaceutically acceptable salt thereof,
(b) Compound B or a pharmaceutically acceptable salt thereof, and
(c) Compound C or a pharmaceutically acceptable salt thereof.

2. The use of the combination of claim 1 for the treatment of a myeloid neoplasm or leukemia.

3. The use of the combination of claim 2, wherein the myeloid neoplasm is a myeloproliferative neoplasm (MPN), a chronic myelogenous leukemia, Chronic neutrophilic leukemia, polycythemia vera (PV), myelofibrosis, primary myelofibrosis (PM), idiopathic myleofibrosis, essential thrombocythemia (ET), Chronic eosinophilic acute leukemia, mastocytosis, a leukemia, MDS, AML, chronic myelogenous leukemia (CML), chronic eosinophilic leukemia, chronic myelomonocytic leukemia, juvenile myelomonocytic leukemia, hypereosinophilic syndrome, systemic rnastocytosis, and atypical chronic myelogenous leukemia.

4. The use of the combination of claim 3 for the treatment of myeloid neoplasm or leukemia with the concurrent or sequential treatment of ruxolitinib, Compound B and Compound C.

5. The use of the combination of claim 1 for the treatment of myelodysplastic syndromes (MDS).

6. A method of treating myeloid neoplasm, leukemia or MDS to a patient, comprising administering a compound of claim 1 to the patient.

7. A pharmaceutical combination comprising
(a) a JAK inhibitor or a pharmaceutically acceptable salt thereof,
(b) a CDK inhibitor or a pharmaceutically acceptable salt thereof, and
(c) a PIM inhibitor or a pharmaceutically acceptable salt thereof.

8. The use of the combination of claim 7 for the treatment of a myeloid neoplasm or leukemia.

9. The use of the combination of claim 8, wherein the myeloid neoplasm is a myeloproliferative neoplasm (MPN), a chronic myelogenous leukemia, Chronic neutrophilic leukemia, polycythemia vera (PV), myelofibrosis, primary myelofibrosis (PM), idiopathic myleofibrosis, essential thrombocythemia (ET), Chronic eosinophilic acute leukemia, mastocytosis, a leukemia, MDS, AML, chronic myelogenous leukemia (CML), chronic eosinophilic leukemia, chronic myelomonocytic leukemia, juvenile myelomonocytic leukemia, hypereosinophilic syndrome, systemic mastocytosis, and atypical chronic myelogenous leukemia.

10. The use of the combination of claim 9 for the treatment of myeloid neoplasm or leukemia with the concurrent or sequential treatment of ruxolitinib, Compound B and Compound C.

11. The use of the combination of claim 7 for the treatment of myelodysplastic syndromes (MDS).

12. A method of treating myeloid neoplasm, leukemia or MDS to a patient, comprising administering a compound of claim 7 to the patient.
